(19) United States
(12) Patent Application Publication
(10) Pub. No.: US 2005/0272712 A1
(43) Pub. Date: Dec. 8, 2005

(54) Compositions and methods for treatment of premenstrual dysphoric disorder

(75) Inventors: Gary Sondermann Grubb, Newtown Square, PA (US); Ginger Dale Constantine, Malvern, PA (US); Karen Walsh, Green Lane, PA (US); Diane Harrison, Villanova, PA (US)

Correspondence Address: WOODCOCK WASHBURN LLP, ONE LIBERTY PLACE - 46TH FLOOR, PHILADELPHIA, PA, 19103, US

(73) Assignee: Wyeth, Madison, NJ (US)

(21) Appl. No.: 11/138,747

(22) Filed: May 26, 2005

Related U.S. Application Data
(60) Provisional application No. 60/574,651, filed on May 26, 2004.

(51) Int. Cl. A61K 31/56

(52) U.S. Cl. 514/170

(57) ABSTRACT

The present invention relates to a method for treating premenstrual dysphoric disorder through administration of at least one progestin and at least one estrogen to a female subject.

### CROSS REFERENCE TO RELATED APPLICATIONS

The present application claims the benefit of U.S. Provisional Application No. 60/574,651, filed May 26, 2004, entitled “Compositions and Methods for Treatments of Premenstrual Dysphoric Disorder,” which is hereby incorporated by reference in its entirety.

### FIELD OF THE INVENTION

In one aspect, the present invention relates to methods for treating premenstrual dysphoric disorder (PMDD) through administration of at least one progestin and at least one estrogen to a female subject.

### BACKGROUND OF THE INVENTION

Premenstrual dysphoric disorder has been estimated to affect 3% to 5% of menstruating women. American Psychiatric Association: Diagnostic and Statistical Manual of Mental Disorder. Fourth Edition, Text Revision. Washington, D.C.: American Psychiatric Association; 2000. PMDD is defined by markedly depressed mood, anxiety, and/or affective lability during the last week of the late luteal phase with absence of these symptoms in the postmenses week. These symptoms can markedly interfere with work or school or with usual social activities and relationships with others. Suppression of ovarian cyclicity is known to alleviate symptoms of PMDD. Freeman et al., J Women Health Gend Based Medi., 10: 561-569, 2001.

Therapeutic interventions for women who meet the criteria for PMDD include selective serotonin reuptake inhibitors (SSRI), tricyclic antidepressants and anxiolytics, as well as the antidepressant alprazolam (XANAX®). These interventions have demonstrated efficacy with minimal side effects. Recent investigations of SSRI have also demonstrated success at low doses. For example, fluoxetine at a daily dose of either 20 mg or 60 mg proved to be superior to placebo in reducing symptoms. Steiner et al., New Engl. J. Med., 332: 1529-34, 1995. An oral dosage formulation of estrogen and progestin for an 81 to 89 day period followed by an oral daily dosage of estrogen for a 2 to 10 day period in female subjects as a contraceptive and for reducing premenstrual symptoms is reported in U.S. Patent Application 2003/0139381. A need exists in the art for alternative treatment regimens that preferably diminish or eliminate premenstrual symptoms including PMDD.

### SUMMARY OF THE INVENTION

In one aspect, the present invention provides methods for treating a female subject suffering from premenstrual dysphoric disorder. Preferred among such methods are those that comprise administering an effective amount of at least one progestin and at least one estrogen to the female subject. In certain embodiments, at least about 4 μg of the at least one progestin (preferably from about 60 to about 120 μg, or more preferably about 90 μg) and/or at least about 1 μg of the at least one estrogen (or preferably from about 15 to about 20 μg, or more preferably about 20 μg) is administered.

The present invention also provides methods comprising administering at least one progestin and at least one estrogen to a female subject daily for at least about 100 days. Preferred methods involve daily administration for at least about 4 months, for at least about 6 months, more preferably at least about 9 months, or even more preferably for at least about 12 months. In certain methods, the female subject suffers from premenstrual dysphoric disorder and the at least one progestin and at least one estrogen are administered in an amount effective for the treatment thereof. In still further methods, at least one progestin and at least one estrogen are administered in an amount effective for contraception.

The present invention also provides kits for treating female subjects, comprising at least about 100 dosage forms that individually comprise at least one progestin and at least one estrogen. In preferred kits, the dosage forms comprises about 90 μg of the at least one progestin and/or about 20 μg of the at least one estrogen. The kits can take the form of, for example, blister packs or other suitable dosage form arrays, and can include at least about 100 such dosage forms, at least about 185 such dosage forms, preferably at least about 275 such dosage forms, or more preferably at least about 365 such dosage forms.

### DETAILED DESCRIPTION OF PREFERRED EMBODIMENTS

Certain methods of the invention involve treating female subject. As used herein, the term “treating” or “treatment” refers to any indicia of success in amelioration of an injury, pathology, or condition, including any objective or subjective parameter such as abatement; inhibition; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. Treating or treatment of any disease or condition disclosed herein includes preventing the onset of symptoms in a subject that may be predisposed to the disease or condition but does not yet experience or exhibit symptoms of the disease or condition (prophylactic treatment), inhibiting the symptoms of the disease or condition (slowing or arresting its development), providing relief from the symptoms or side-effects of the disease or condition (including palliative treatment), and/or relieving the symptoms of the disease or condition (causing regression). Accordingly, the term “treating” includes the administration of compounds or agents to a subject to prevent or delay, to alleviate, or to arrest or inhibit development of the symptoms or conditions associated with a disease state. A skilled medical practitioner will know how to use standard methods to determine whether and to what extent a patient is suffering from premenstrual dysphoric disorder. Such a determination can be made before administration of an effective amount of progestin and estrogen and/or after administration.

Preferred methods of the invention involve administering an effective amount of at least one progestin and at least one estrogen to a female subject. The term “progestin,” as used herein, refers to any progestationally active compound, i.e., any compound that binds to and activates any progesterone receptor. Representative progestins include progesterone synthetic derivatives such as, for example, 17-hydroxy progesterone esters, 19-nor-17-hydroxy progesterone esters, 17α-ethinyltestosterone and derivatives thereof, 17α-ethinyl-19-nor-testosterone and derivatives thereof, norethindrone, norethindrone acetate, ethynodiol diacetate, dydrogesterone, medroxy-progesterone acetate, norethynodrel, allylestrenol, lynoestrenol, fuingestanol acetate, medrogestone, norgestrienone, dimethiderome, ethisterone, cyproterone acetate, levonorgestrel, dl-norgestrel, d-17α-acetoxy-13β-ethyl-17a-a-ethinyl-gon-4-en-3-one oxime, cyproterone acetate, gestodene, desogestrel, etonorgestrel, norgestimate and norelgestromin. Other compounds with progestational activity used in oral contraceptives include chlormadione, dienogest, and drospirenone. One preferred progestin is levonorgestrel.

The term “estrogen,” as used herein, refers to a group of synthetic or natural estrogens, including steroidal and nonsteroidal estrogens. The natural estrogens can be mammalian-derived or plant-derived. In humans, estrogens are formed in the ovary, possibly the adrenal cortex, the testis, and the fetoplacental unit and have various functions in both sexes. Estrogen is included within a class of ovulation inhibitors to prevent breakthrough (mid-cycle) bleeding during the ovulation cycle. The ring system of an estrogen is estrane, an 18-carbon tetracyclic hydrocarbon nucleus that is the parent structure of the estrogenic steroids. Estrogens typically have an aromatic A ring with a phenolic 3-OH group and an oxygen function on C17. Estrogens are defined as any compound that binds to and activates any estrogen receptor. The synthetic estrogens can be for example, ethinyl estradiol, ethynodiol diacetate, mestranol and quinestranol. Particularly of interest are 17α-ethinyl estradiol and esters and ethers thereof. One preferred estrogen is 17α-ethinyl estradiol. The natural estrogens can include, for example, conjugated equine estrogens, esterified estrogens, 17β-estradiol, estradiol valerate, estrone, piperazine estrone sulphate, estriol, estriol succinate and polyestrol phosphate. Other useable estrogens include the esters of estradiol, estrone and ethinyl estradiol such as the acetate, sulfate, valerate or benzoate, conjugated equine estrogens, agonist anti-estrogens, and selective estrogen receptor modulators.

The progestins and estrogens of the invention can be administered in any amount effective to treat premenstrual dysphoric disorder, and/or to achieve contraception. In a preferred embodiment, at least about 4 μg of at least one progestin, for example, levonorgestrel (preferably from about 4 to about 120 μg, more preferably from about 60 to about 110 μg, or more preferably about 90 μg) and at least about 1 μg of at least one estrogen, for example, ethinyl estradiol, (preferably from about 1 to about 20 μg, more preferably from about 15 to about 20 μg, or more preferably about 20 μg) is administered. It is preferred that the progestin dosage be not greater than 120 μg per day (when levonorgestrel is used), and that the estrogen dosage be not greater than 20 μg per day (when ethinyl estradiol is used). It is also preferred that the progestin and estrogen be administered at a constant, or at least relatively constant, daily dosage.

Although administration of ethinyl estradiol at a dosage of approximately 20 μg per day and levonorgestrel at a dosage of approximately 90 μg per day is preferred, one can use at least about 1 μg of ethinyl estradiol, (preferably from about 1 to about 20 μg, more preferably from about 15 to about 20 μg, or more preferably about 20 μg), and at least about 4 μg of levonorgestrel (preferably from about 4 to about 120 μg, more preferably from about 60 to about 110 μg, or more preferably about 90 μg). Other estrogens and progestins vary in potency from ethinyl estradiol and levonorgestrel, respectively. To the extent that other estrogens are used, either alone or in combination with ethinyl estradiol, it is preferred that the amount of estrogen used correspond to the stated amounts of ethinyl estradiol. Similarly, to the extent that other progestins are used, either alone or in combination with levonorgestrel, it is preferred that the amount of progestin used correspond to the stated amounts of levonorgestrel. The correlations in potency between the various estrogens and progestins are generally known to those skilled in the art, see, e.g., European Patent Application No. 0 253 607; U.S. Application No. 2003/0139381, each incorporated herein by reference in their entirety and for all purposes.

The methods of the invention preferably involve administering progestin and estrogen daily for at least about 100 days. In certain embodiments, administration is daily for at least about 4 months daily, for at least about 6 months, daily for at least about 9 months, and/or daily for at least about 12 months. Certain methods of the invention involve a so-called twenty-eight day regimen that involves administering estrogen and progestin, preferably monophasicly, for 28 consecutive days. The 28-day treatment cycles are continued for multiple cycles to provide a constant dosage of estrogen and progestin for up to 6 months, up to 12 months, up to 18 months, up to 24 months or longer. In preferred embodiments, women are administered an oral contraceptive on days 1 through 28 of the menstrual cycle containing 90 μg levonorgestrel and 20 μg ethinyl estradiol per day. Thus, in a 28-day regimen schedule, there are about 13 treatment cycles per year, thus limiting or eliminating all menstrual cycles per year. The treatment regimen can be continued for an extended administration period, for example, one year or longer, or two years or longer.

The formulations of the invention can be administered orally, parenterally, sublingually, transdermally, topically, intravaginally, intranasally or buccally in a variety of suitable dosage forms. The method of administration depends on the types of estrogens and progestins used, as well as the amounts per unit dosage. Pharmaceutical formulations or preparations containing the formulations of the invention and a suitable carrier can be solid dosage forms which includes tablets, dragees, capsules, cachets, pellets, pills, powders or granules; topical dosage forms which include solutions, powders, fluid emulsions, fluid suspensions, semi-solids, ointments, pastes, creams, gels or jellies, foams and controlled release depot entities; transdermals, vaginal rings, buccal formulations; and parenteral dosage forms which includes solutions, suspensions, emulsions or dry powder comprising an effective amount of estrogen, progestin and antidepressant as taught in this invention. “Depot” or “drug depot” refers to a reservoir containing a composition that is implanted into, or in some fashion connected to a patient such that the compound is delivered to the patient. The depot may or may not regulate the administration of the compound.

Pharmaceutically acceptable carriers are determined in part by the particular composition being administered, as well as by the particular method used to administer the composition. Accordingly, there is a wide variety of suitable formulations of pharmaceutical compositions for administering the hormonal contraceptive product. It is known in the art that active ingredients can be contained in such formulations in addition to pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. See, e.g., Remington's Pharmaceutical Sciences, Mack Publishing Co., Easton, Pa. 18th ed., 1990; Modern Pharmaceutics, Banker & Rhodes, Marcel Dekker, Inc., 1979; or Goodman & Gilman's The Pharmaceutical Basis of Therapeutics, 6th Edition, MacMillan Publishing Co., New York, 1980, each incorporated herein by reference in their entirety and for all purposes. The pharmaceutical compositions are generally formulated as sterile, substantially isotonic and in full compliance with all Good Manufacturing Practice (GMP) regulations of the U.S. Food and Drug Administration.

Generally speaking, the formulations are prepared according to conventionally known procedures in accordance with the method of administration. Thus, the active ingredients are prepared according to known methods in a pharmaceutically acceptable form for administration. These ingredients, in their required quantities are combined with the appropriate pharmaceutical carriers such as additives, vehicles and/or flavor ameliorating substances. These substances can be referred to as diluents, binders and lubricants. Gums, starches and sugars are also common terms. Typical of these types of substances or excipients are pharmaceutical grades of mannitol, lactose starch, magnesium stearate, sodium saccharin, talcum, cellulose, glucose, sucrose, magnesium carbonate and the like. The active ingredient(s) can comprise from about 0.01% by weight to about 99.99% by weight of the total formulation and the remainder comprises the pharmaceutically acceptable carrier. The percentage of active ingredient(s) can vary according to the delivery system or method of administration and is chosen in accordance with conventional methods known in the art.

Most estrogens are orally active and that route of administration (preferably in tablet or capsule form) is therefore preferred. Pharmaceutical dosage forms for oral use can be obtained through combination of the compounds of the present invention with a solid excipient, optionally grinding a resulting mixture, and processing the mixture of granules, after adding suitable additional compounds, if desired, to obtain tablets or cores. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Methods for transdermal administration, including the associated manufacturing methods, are well known in the art. In this connection, reference may be had to U.S. Pat. Nos. 4,752,478, 4,685,911, 4,438,139 and 4,291,014, each incorporated herein by reference in their entirety and for all purposes.

Dosage forms according to the invention can be placed in an appropriate package and labeled for treatment. Such packages (whether in the form of blister packs, tablet dispensers, or the like) are referred to herein as kits, typically include the daily dosages arranged for proper sequential administration. Preferred kits contain multiple dosage forms in a synchronized, fixed sequence, wherein the sequence or arrangement thereof corresponds to the stages of daily administration. For example, dosage forms can be provided in kit form containing about 18 to about 28 tablets for a 28-day regimen, preferably about 21 to about 28 tablets. These tablets are intended for ingestion on successive days. For a more long-term regimen, the dosage forms can be provided in kit form containing at least about 60 tablets, and preferably at least about 81 to 89 tablets, and up to 110 tablets, intended for ingestion on successive days. Preferably administration is daily for at least 100 days. Daily administration for at least 168 days, for at least 336 days, or for a year or longer can also be effected. For administration of multiple dosage forms from a kit, the provided labeling will typically include, for example, instructions concerning the amount, frequency and method of administration of each dosage form. Preferred kits are those that include at least 100 dosage forms that individually include at least one progestin and at least one estrogen. Such kits can, in certain embodiments, include at least about 185 of the dosage forms, at least about 275 of the dosage forms, and/or at least about 365 of the dosage forms.

Although we do not wish to be bound by any particular theory or mechanism of action, it is believed that the treatment regimens of the present invention suppress the hypothalamic-pituitary-ovarian axis without hypoestrogenemia. It is believed that the combination of estrogen and progestin at a constant dosage suppresses both endogenous and exogenous hormonal fluctuations, as well as ovarian activity and the production of cyclic estrogen, progesterone, luteinizing hormone, and follicle-stimulating hormone.

The methods of the invention can be evaluated for their effect on premenstrual symptomatology using, for example, psychometric scales that include self-administered Visual Analogue Scales (VAS) and a prospective daily symptoms chart or diary to evaluate psychological and somatic symptoms. Total score of the psychological and somatic symptoms is computed. The VAS measures tension, irritability, dysphoria, sleeping and eating patterns, headache, bloating, pain and breast tenderness and weight gain symptoms.

### EXAMPLES

The invention is further demonstrated in the following examples. The examples are for purposes of illustration and are not intended to limit the scope of the present invention.

### Example 1

The levonorgestrel/ethinyl estradiol (LNG/EE) dose chosen for the study is based on ovarian suppression studies that were used to estimate the degree of ovarian suppression with low-dose continuous LNG/EE regimens. Results of these studies demonstrate that although ovulation is suppressed at doses≧LNG 90 μg/EE 15 μg in 24-day regimens, ovarian activity was evident with the LNG 90 μg/EE 15 μg dose. This suggests that additional estrogen, to suppress follicle stimulating hormone, will be beneficial. Additionally, this series of studies showed better ovarian suppression with the 24-day regimen than the 21-day regimen. It is expected that extending pill-taking to a continuous regimen, e.g., daily administration for 100 days or more, will further suppress ovarian activity. Therefore, LNG 90 μg/EE 20 μg is the dose selected for the continuous clinical program as it is expected to provide excellent contraceptive efficacy at a low daily dose.

### Example 2

A Multicenter, Randomized, Double-Blind, Placebo-Controlled Study of a Combination Regimen of Levonorgestrel and Ethinyl Estradiol in a Continuous Daily Regimen

Trial Design

A phase 3, multicenter, randomized, double-blind, placebo-controlled study to be conducted at approximately 75 sites. The primary endpoint is to evaluate the effect of treatment with LNG/EE administered in a continuous daily regimen versus placebo on the mean change in average Daily Record of Severity of Problems (DRSP) 21-item total daily scores from baseline to the last on-therapy efficacy period. The efficacy period for each on-therapy cycle will be defined on an individual subject basis. Each subject's average cycle length will be calculated using the pretreatment screening cycle 2 and placebo run-in cycle 3 data. The secondary endpoints are to evaluate the effect of treatment with LNG/EE administered in a continuous daily regimen versus placebo on the following:
Mean change from baseline in Clinical Global Impression-Severity (CGI-S) scores.
Responder analyses based on CGI-S scores, percentage improvements in DRSP scores and PMDD criteria.
Change from baseline in mean clinically defined DRSP cluster (symptom subgroup) scores.
Change from baseline in Work Limitations Questionnaire (WLQ).
Area under the curve (AUC) analysis of DRSP scores for the entire 112-day period.
Subject global evaluation mean scores.
Change in weight.

The first 2 cycles of the study will be pretreatment screening cycles, followed by 1 cycle of single-blind placebo run-in treatment. Thereafter, four 28-day pill packs of double-blind treatment will be followed by a posttreatment visit. Subject study participation will be approximately 8 months.

In order to be randomly assigned to the double-blind treatment phase of this study, a subject must meet the DRSP inclusion criteria during both the pretreatment screening cycle 2 and the placebo run-in cycle 3. The mean change in DRSP scores from the baseline efficacy period to the last on-therapy efficacy period is of primary interest, although change from baseline during each double-blind treatment cycle will be examined as well.

During the double-blind treatment interval, each subject will be randomly assigned to receive either tablets containing LNG 90 μg and EE 20 μg or matching placebo. With the exception of the beginning of cycle 4, subjects will take 1 pill daily, orally, for approximately 112 days at approximately the same time each day.

1. A method for treating a female subject suffering from premenstrual dysphoric disorder comprising administering an effective amount of at least one progestin and at least one estrogen to said female subject, wherein said effective amount is administered daily for at least about 100 days.

2. The method of claim 1 wherein said female subject suffers from premenstrual dysphoric disorder and said effective amount is effective for treating premenstrual dysphoric disorder.

3. The method of claim 1 wherein at least about 4 μg of levonorgestrel, or a progestin dosage of corresponding potency, is administered.

4. The method of claim 1 wherein from about 60 μg to about 110 μg of levonorgestrel, or a progestin dosage of corresponding potency, is administered.

5. The method of claim 1 wherein at least about 1 μg of ethinyl estradiol, or an estrogen dosage of corresponding potency, is administered.

6. The method of claim 1 wherein from about 15 μg to about 20 μg of ethinyl estradiol, or an estrogen dosage of corresponding potency, is administered.

7. The method of claim 1 wherein said at least one progestin is chlormadinone acetate, norethisterone acetate, cyproterone acetate, desogestrel, gestodene, drospirenone, etonorgestrel, norgestimate, norelgestromin, or levonorgestrel.

8. The method of claim 1 wherein said at least one estrogen is ethinyl estradiol, mestranol, estradiol, estriol, estrone, or estrane.

9. The method of claim 7 wherein said at least one progestin is levonorgestrel administered at a dosage not greater than 90 μg per day, or a progestin dosage of corresponding potency is administered.

10. The method of claim 8 wherein said at least one estrogen is ethinyl estradiol administered at a dosage not greater than 20 μg per day, or an estrogen dosage of corresponding potency is administered.

11. The method of claim 1 wherein said at least one estrogen is ethinyl estradiol administered at a dosage of approximately 20 μg per day, and said at least one progestin is levonorgestrel administered at a dosage of approximately 90 μg per day, or an estrogen dosage of corresponding potency and a progestin dosage of corresponding potency is administered.

12. The method of claim 1 wherein said effective amount is administered orally, transdermally, or via depot to the subject.

13. The method of claim 1 wherein said effective amount is administered daily for at least about 4 months.

14. The method of claim 1 wherein said effective amount is administered daily for at least about 6 months.

15. The method of claim 1 wherein said effective amount is administered daily for at least about 9 months.

16. The method of claim 1 wherein said effective amount is administered daily for at least about 12 months.

17. The method of claim 1 wherein said effective amount is administered in a dosage form.

18. The method of claim 1 wherein said dosage form is a tablet or capsule.

19. The method of claim 17 wherein said dosage form is administered orally, transdermally or via depot to the subject.

20. The method of claim 1 further comprising determining an extent to which said female subject suffers from premenstrual dysphoric disorder.

21. The method of claim 20 wherein said determination is made before said administering step.

22. The method of claim 20 wherein said determination is made after said administering step.

23. A method comprising administering at least one progestin and at least one estrogen to a female subject daily for at least about 100 days.

24. The method of claim 23 wherein said female subject suffers from premenstrual dysphoric disorder and said at least one progestin and at least one estrogen are administered in an amount effective for treating premenstrual dysphoric disorder.

25. The method of claim 23 wherein at least about 4 μg of levonorgestrel, or a progestin dosage of corresponding potency, is administered.

26. The method of claim 23 wherein from about 60 μg to about 110 μg of levonorgestrel, or a progestin dosage of corresponding potency, is administered.

27. The method of claim 23 wherein at least about 1 μg of ethinyl estradiol, or an estrogen dosage of corresponding potency, is administered.

28. The method of claim 23 wherein from about 15 μg to about 20 μg of ethinyl estradiol, or an estrogen dosage of corresponding potency, is administered.

29. The method of claim 23 wherein said at least one progestin is chlormadinone acetate, norethisterone acetate, cyproterone acetate, desogestrel, gestodene, drospirenone, etonorgestrel, norgestimate, norelgestromin, or levonorgestrel.

30. The method of claim 23 wherein said at least one estrogen is ethinyl estradiol, mestranol, estradiol, estriol, estrone, or estrane.

31. The method of claim 29 wherein said at least one progestin is levonorgestrel administered at a dosage not greater than 90 μg per day, or a progestin dosage of corresponding potency is administered.

32. The method of claim 30 wherein said at least one estrogen is ethinyl estradiol administered at a dosage not greater than 20 μg per day, or an estrogen dosage of corresponding potency is administered.

33. The method of claim 23 wherein said at least one estrogen is ethinyl estradiol administered at a dosage of approximately 20 μg per day, and said at least one progestin is levonorgestrel administered at a dosage of approximately 90 μg per day, or an estrogen dosage of corresponding potency and a progestin dosage of corresponding potency is administered.

34. The method of claim 32 wherein said at least one progestin and at least one estrogen are administered orally, transdermally, or via depot to the subject.

35. The method of claim 23 wherein said at least one progestin and at least one estrogen are administered daily for at least about 6 months.

36. The method of claim 23 wherein said at least one progestin and at least one estrogen are administered daily for at least about 9 months.

37. The method of claim 23 wherein said at least one progestin and at least one estrogen are administered daily for at least about 12 months.

38. The method of claim 23 wherein said at least one progestin and at least one estrogen are administered in an amount effective for contraception.

39. A kit for treating a female subject comprising at least about 100 dosage forms that individually comprise at least one progestin and at least one estrogen.

40. The kit of claim 39 wherein each of said dosage forms comprise at least about 4 μg of levonorgestrel or a progestin dosage form of corresponding potency.

41. The kit of claim 39 wherein each of said dosage forms comprise from about 60 μg to about 110 μg of levonorgestrel or a progestin dosage form of corresponding potency.

42. The kit of claim 39 wherein each of said dosage forms comprise at least about 1 μg of ethinyl estradiol or an estrogen dosage form of corresponding potency.

43. The kit of claim 39 wherein each of said dosage forms comprise from about 15 μg to about 20 μg of ethinyl estradiol or an estrogen dosage form of corresponding potency.

44. The kit of claim 39 wherein said at least one progestin is progesterone, chlormadinone acetate, norethisterone acetate, cyprotherone acetate, desogestrel, drospirenone, etonorgestrel, norgestimate, norelgestromin, or levonorgestrel.

45. The kit of claim 39 wherein said at least one estrogen is ethinyl estradiol, mestranol, estradiol, estriol, estrone, or estrane.

46. The kit of claim 44 wherein each of said dosage forms comprise levonorgestrel in an amount no greater than 90 μg or a progestin dosage form of corresponding potency.

47. The kit of claim 45 wherein each of said dosage forms comprise ethinyl estradiol in an amount no greater than 20 μg or an estrogen dosage form of corresponding potency.

48. The kit of claim 39 wherein each of said dosage forms comprise approximately 20 μg ethinyl estradiol and approximately 90 μg levonorgestrel, or an estrogen dosage form and a progestin dosage form of corresponding potency.

49. The kit of claim 39 that comprises at least about 185 of said dosage forms.

50. The kit of claim 39 that comprises at least about 275 of said dosage forms.

51. The kit of claim 39 that comprises is at least about 365 of said dosage forms.

52. The kit of claim 39 wherein said dosage forms are tablets, capsules, or a combination thereof.
